(19) United States
(12) Patent Application Publication
(10) Pub. No.: US 2002/0133224 A1
(43) Pub. Date: Sep. 19, 2002

(54) Drug eluting encapsulated stent

(76) Inventors: Clara Bajgar, Arlington, MA (US); Michael Szycher, Lynnfield, MA (US)

Correspondence Address: FOLEY HOAG LLP, PATENT GROUP, 155 SEAPORT BOULEVARD, BOSTON, MA, 02110, US

(21) Appl. No.: 10/092,177

(22) Filed: Mar. 6, 2002

Related U.S. Application Data
(60) Provisional application No. 60/275,504, filed on Mar. 13, 2001.

(51) Int. Cl. A61F 2/06

(52) U.S. Cl. 623/1.39; 623/1.43

(57) ABSTRACT

A stent substantially completely encapsulated with a microporous polymeric membrane is provided. Encapsulation of the stent may be accomplished by an electrostatic deposition process. The microporous polymeric membrane may contain variable concentrations of one or more pharmacotherapeutic agents. After deployment to a site of interest, the stent and more specifically, the membrane, provides local delivery of sustained or controlled therapeutic dose of one or more of suitable pharmacotherapeutic agent.

### RELATED U.S. APPLICATION(S)

This application claims priority to U.S. Provisional Application Serial No. 60/275,504, filed Mar. 13, 2001, which application is hereby incorporated herein by reference.

### FIELD OF THE INVENTION

The present invention relates to devices and methods for local drug delivery to intravascular sites, and more particularly, to devices and methods for treatment of restenosis following, for instance, balloon angioplasty.

### BACKGROUND OF THE INVENTION

Currently, methods for preventing or controlling restenosis are specifically aimed at influencing factors believed to be involved in the body's response to external or internal tissue stimulants, such as angioplasty, stenting procedures, and/or viruses. Common countermeasures which have been used to prevent or control restenosis generally fall into the one of several categories, including (1) mechanical atheroablative techniques, such as debulking, vascular filters, and emboli-trapping devices, (2) ultrasound-initiated atheroablative techniques, (3) light-assisted procedures, predominantly excimer laser angioplasty, (4) pharmacological agents and gene therapy, (5) ultraviolet photophoresis, believed to be an immune modulator, (6) radiation therapy, such as external and endovascular brachytherapy, and (7) re-stenting.

In spite of advances in each of these individual technological areas, restenosis continues to be a problem.

Stents

Stents are small mechanical devices which can be implanted into a blood vessel to prevent re-narrowing or closure of a vessel opened during angioplasty. Typically, a stent comprising a mesh or perforated tube can be inserted directly to the site of closure or narrowing, and can be mechanically expanded by, for instance, a balloon to reopen the vessel at the site of closure. The mechanical reopening of the vessel with a balloon can sometimes lead to balloon-related injuries to the tissues at the site of closure. Such injuries can often stimulate tissue proliferation at the reopened site during the healing process, and which proliferation can result in pronounced neointimal hyperplasia or restenosis. Restenosis remains the most common post-stenting clinical problem, and requires effective intervention or counter-measures to prevent and/or control its reoccurrence.

To prevent and/or control restenosis, modifications to stent designs and materials have been proposed, and in some instances, evaluated. One of several new approaches is the development of non-metallic, biodegradable stent materials, such as high molecular weight Poly-1-lactic acid (PLLA).

In addition, numerous inorganic coatings and surface treatments have been developed to improve chemical inertness and biocompatibility of metallic stents. Some organic coatings, such as gold, however, yield a higher rate of in-stent restenosis than uncoated stents. Others, including silicon carbide and turbostatic carbon, show promise and are currently in clinical trials. It has been observed that electrochemical polishing of stainless steel stents can result in decreased blood clot formation, and can lower neointimal hyperplasia in porcine models. (Erbel et al., Alternative Methods in Interventional Therapy of Coronary Heart Disease, Z. Kardiol. 1995, 84 Suppl 2: 53-64; Gutensohn et al., In Vitro Analysis of Diamond-like Carbon Coated Stents. Reduction of Metal Ion Release, Platelet Activation, and Thrombogenicity, Thromb. Res. 2000, Sept. 99(6):577-585; De Scheerder et al., Neointimal Hyperpasia of Corornary Stents, J. Interv. Cardiol. 2000, 13: 179-186; Tanigawa et al., Reaction of the Aortic Wall to Six Metallic Stent Materials, Acad. Radiol. 1995, 2(5): 379-384; Hehrlein et al., Influence of Surface Texture and Charge on the Biocompatibility of Endovascular Stents, Coron. Artery Dis. 1995, 6(7):581-586).

Organic coatings, including both synthetic and natural coatings, have also been widely studied. Among the synthetic coatings studied are Dacron, polyester, polyurethane, polytetrafluoroethylene (PTFE), polyethylacrylate/polymethylmetahcrylate, polyvinyl chloride, silicone, collagen, and iridium oxide. Results of studies, such as those with PTFE-coated stents, are disappointing or mixed at best, as there are high occurrences of late thrombo-occlusive events. With only a very few exceptions, the general consensus is that any favorable outcome was not associated with treatment of conventional in-stent restenosis using PTFE-coated stents. (Makutani et al., Effect of Antithrombotic Agents on the Patency of PTFE-Covered Stents in the Inferior Vena Cava: An Experimental Study, Cardiovasc. Intervent. Radiol. 1999, 22: 232-238; Farber et al., Access-Related Venous Stenoses and Occlusions: Treatment with Percutaneous Transluminal Angioplasty and Dacron-Covered Stents, Cardiovase. Intervent Radiol. 1999, 22: 214-218; Costamagna et al., Hydropgilic Hydromer-Coated Polyurethane Stents Versus Uncoated Stents in Malignant Biliary Obstruction: A Randomized Trial, Gastrointest. Endosc. 2000, 51(1):8-11; Whealan et al., Biocompatibility of Phosphorylcholine Coated Stents in Porcine Coronary Arteries, Heart2000, 83(3):338-345; Zheng et al., Clinical Experience with a New Biocompatible Phosphorylcholine Coated Coronary Stent, J. Invas. Cardiol. 1999, 11(10):608-614; Bar et al., New Biocompatible Polymer Surface Coating for Stents Results in a Low Neointimal Response, J. Biomed. Mater. Res. 2000, 52(1):193-8; Rechavia et al., Biocompatibility of Polyurethane-Coated Stents: Tissue and Vascular Aspects, Cathet. Cardiovasc. Diagn. 1998, 45(2):202-207; Dev et al., Kinetics of Drug Delivery to the Arterial Wall via Polyurethane-Coated Removable Nitinol Stent: Comparative study of Two Drugs, Cathet. Cardiovasc. Diagn. 1995, 34(3):272-278; Dolmatch et al., Patency and Tissue Response Related to two Types of Polytetrafluoroethylene-Coated Stents in the Dog, J Vasc. Interv. Radiol. 1996, 7(5):641-649; Tepe et al., Covered Stents for Prevention of Restenosis. Experimental and Clinical Results with Different Stent Designs, Invest. Radiol. 1996, 31(4):223-229; Briguori et al., Polytetrafluoroethylene-covered Stents for the Treatment of Narrowings in Aorticocoronary saphenous Vein Grafts, Am. J. Cardiol. 2000, 86(3):343-346).

An autologous arterial graft covering the external surface of a conventional stent in porcine models, on the other hand, has been observed to perform nicely, resulting in accelerated endothelialization, less vascular injury, less thinning of the arterial media, and a trend toward reducing intimal hyperplasia in normal coronary arteries. Such a result has prompted additional studies into the usefulness of providing an encapsulated stent (i.e., stent with a covering). (Stefanadis et al., Stents Covered by an Autologous Arterial Graft in Porcine Coronary Arteries: Feasibility, Vascular Injury and Effect on Neointimal Hyperplasia, Cardiovasc. Res. 1999, 41(2)432-442; Marin et al., Effect of Polytetrafluoroethylene Covering of Palmaz Stents on the Development of Intimal Hyperplasia in Human Iliac Arteries, J. Vasc. Interv. Radiol. 1996, 7(5):651-656).

The term “coated stent” refers to a stent in which its metallic mesh may be coated with a biocompatible or biodegradable layer that is suitable for use as a drug carrying layer. It should be noted that passages in the body of a coated stent (i.e., the openings within the mesh) remain fully open and are not covered with a layer of the coating.

Coated stents are usually prepared by a process involving immersion coating and aerosol spraying of the drug loaded material onto the coating. Variations to this process include attaching a pre-existing membrane and embedding the drug loaded material on the surface by ion bombardment.

The term “covered stent” refers to a stent in which the stent structure, both the metal mesh support and the openings defined by the struts (i.e., openings within the mesh), are completely covered with the same biocompatible non-porous material. However, the cover is non-porous and contains no drugs. Such a stent is not a drug-eluting stent.

Pharmacotherapeutics

Intracoronary intervention can reduce neointima formation by reducing smooth muscle cell proliferation after balloon angioplasty. However, such intervention is often complicated by subacute and late thrombosis. Coronary thrombo-aspiration and coronary pulsed-spray procedures, followed by immediate endovascular therapy, have been particularly helpful in removing thrombotic material associated with plaque. Histologic analysis of in-stent restenosis has shown that thrombus is present in less than five percent of the area, inflammatory cells are present in fifteen percent of cells (ten percent leukocytes), smooth muscle cells account for fifty-nine percent of cells, activated smooth muscle cells comprise twenty five percent, and apoptosis afflicts twelve percent. (Ettenson et al., Local Drug Delivery: An Emerging Approach in the Treatment of Restenosis, Vasc. Med. 2000, 5(2):97-102; Camenzind E., Local Vascular Therapy Against Thrombus and Proliferation: Clinical Trials Update, American College of Cardiology 1998; Gonschior P., Local Drug Delivery for Restenosis and Thrombosis - Progress, J. Invas. Cardiol. 1998, 10(8):528-532).

Pharmacotherapeutic agents have been used for the treatment of some of the major post-angioplasty complications, including immunosuppresants, anticoagulants and anti-inflammatory compounds, chemotherapy agents, antibiotics, antiallergenic drugs, cell cycle inhibitors, gene therapy compounds, and ceramide therapy compounds. Pharmacotherapeutic agents can be delivered either systemically or locally. Systemic treatment has shown limited success in reducing restenosis following stent implantation, a result believed to be due to inadequate concentration of the pharmacotherapeutic agents at the site of injury. Increased dose administration, however, is constrained by possible systemic toxicity. It has been observed that local delivery of higher doses via drug eluting stents can significantly reduce adverse systemic effects. (Raman et al., Coated Stents: Local Pharmacology, Semin. Interv. Cardiol. 1998, 3(3-4):133-137).

Heparin and glycosaminoglycans are examples of anticoagulants which interact with growth factors and other glycoproteins. In several animal models, heparin, delivered locally after stent implantation, has not been observed to reduce neointimal proliferation. In 1998,the Total Occlusion Study of Canada, was initiated to determine, in a randomized trial on 410 patients, whether clinical outcome following successful PTCA of totally occluded arteries can be improved by the use of a heparin-coated stents. (Nelson et al., Endovascular Stents and Stent-Grafts: Is Heparin Coating Desirable?, Cardiovasc. Intervent. Radiol. 2000, 23(4):252-255; Baumbach et al., Local Delivery of a Low Molecular Weight Heparin Following Stent Implantation in the Pig Corornary Artery, Basic Res. Cardiol. 2000, 95(3):173-178; Ahn et al., Preventive Effects of the Heparin-Coated Stent on Restenosis in the Porcine Model, Catheter Cardiovasc. Interv. 1999, 48(3):324-330; Dzavik et al., An Open Design, Multicentre, Randomized Trial of Percutaneous Transluminal Coronary Angioplasty Versus Stenting, with a Heparin-Coated Stent, of Totally Occluded Corornary Arteries: Rationale, Trial Design and Baseline Patient Characteristics. Total Occlusion Study of Canada Investigators. Can. J Cardiol. 1998, 14(6):825-832).

Abciximab is a genetically engineered fragment of a chimeric human-murine mono-clonal antibody. It is a glycoprotein inhibitor, and works by inhibiting the binding of fibrinogen and other substances to glycoprotein receptor (GBIIb/IIIa) on blood platelets integral to aggregation and clotting. Abciximab appears to be effective in preventing platelet aggregation when used with aspirin and heparin, and appears to be effective in preventing abrupt closure of arteries. (Aristides et al., Effectiveness and Cost Effectiveness of Single Bolus Treatment with Abciximab (Reo Pro) in Preventing Restenosis Following Percutaneous Transluminal Coronary Angioplasty in High Risk Patients, Heart 1998, 79(1):12-17).

Dexamethasone, an anti-inflammatory drug, has failed to reduce neointimal hyperplasia in a majority of cases. It has been reported that Pemirolast Potassium, an antiallergic drug, inhibits post-PTCA restenosis in animal experiments. (Lincoff et al., Sustained Local Delivery of Dexamethasone by a Novel Intravascular Eluting Stent to Prevent Restenosis in the Porcine Corornary Injury Model, J. Am. Coll. Card. 1997, 29(4):808-816; Ohsawa et al., Preventive Effects of an Antiallergic Drug, Pemirolast Potassium, on Restenosis After Percutaneous Transluminal Corornary Angioplasty, Am. Heart J. 1998, 136(6):1081-1087).

In the group of cancer treatment drugs, Paclitaxel, a potent anti-neoplastic compound, was found to reduce neointima. Taxol-based studies were essential in suggesting the role of growth-regulatory molecules in vascular smooth muscle cell proliferation. Clinical trials evaluating the safety and effectiveness of Paclitaxel-coated coronary stents have recently been completed. (Herdeg et al., Paclitaxel: a Chemotherapeutic Agent for Prevention of Restenosis? Experimental Studies in Vitro and in Vivo, Z. Kardiol. 2000, 89(5):390-397; Herdeg et al., Local Paclitaxel Delivery for the Prevention of Restenosis: Biological Effects and Efficacy in Vivo, J. Am. Coll. Cardiol. 2000, 35(7):1969-1976).

The exact role of antibiotics in treatment of coronary artery disease has not been fully established. It is known that antibiotics are effective in controlling inflammation caused by a variety of infectious agents found in fatty plaques blocking the arteries. Results of clinical investigation with azithromycin suggest only modest antibiotic benefits for heart patients. Findings are sufficiently promising to warrant continuing research with several different types of antibiotics, including Rapamycin.

Gene therapy for restenosis has been directed towards smooth muscle cells and involves gene transfer via DNA, with or without integration of chromosomes, into selected cells. In transduction without integration, the gene is delivered to both cytoplasm and nucleus and is therefore non-selective. Gene transfer for integration employs retrovirus to affect growth stimulators. (Nikol et al., Gene Therapy for Restenosis: Progress or Frustration?, J. Invas. Cardiol. 1998, 10(8):506-514).

Recent studies with ceramides show a marked decrease in neointimal hyperplasia following stretch injury in carotid arteries in rabbit models. One of the more widely researched antibiotics from this category is Rapamycin, a phospholipid exhibiting immunosuppressive properties. It has been shown to block T-cell activation and proliferation, inhibit Taxol-induced cell cycle apoptosis, and activate protein kinase signal translation in malignant myogenic cells. Rapamycin and its analogs exhibit anti-tumor activities at relatively low dose levels, while inducing only mild side effects, an extremely important aspect of patient care. (Story et al., Signal Transduction During Apoptosis; Implications for Cancer Therapy, Frontiers in Bioscience, 1998, 3: 365-375; Calastretti et al., Taxol Induced Apoptosis and BCL-2 Degradation Inhibited by Rapamycin, Suppl. to Clinical Cancer Research, 1999, Vol 5; Shu et al., The Rapamycin Target, mTOR Kinase, May Link IGF-1 Signaling to Terminal Differentiation, Proc. Amer. Assoc. Cancer Res. 40, 1999; Shikata et al., Kinetics of Rapamycin-Induced Apoptosis in Human Rhabdomyosarcoma Cells, Proc. Amer. Assoc. Cancer Res. 40, 1999; Sekulic et al., A Direct Linkage Between the Phosphoinositide 3-Kinase-AKT Signaling Pathway and the Mammalian Target of Rapamycin in Mitogen-Stimulated and Transformed Cells, Cancer Research, 2000, 60: 3504 13; Vasey P., Clinical Trials: New Targets, New Agents, American Society of Clinical Oncology 36th Annual Meeting, May 2000; Murphy B., T-Cell Triggering and Transduction, American Society of Transplantation4 th Annual Winter Symposium, January. 2000; Charles et al., Ceramide-Coated Balloon Catheters Limit Neointimal Hyperplasia after Strech Injury in Carotid Arteries, Integrative Physiology, Circulation Research, August. 2000, 87: 282).

### SUMMARY OF THE INVENTION

The present invention provides, in one embodiment, an encapsulated stent for local delivery of at least one pharmacotherapeutic agent to an intravascular site, for the treatment of, for instance, restenosis following, for example, balloon angioplasty.

The stent, in accordance with an embodiment of the invention, includes a substantially cylindrical hollow body, a membrane positioned about a periphery of the body, and a plurality of pores throughout the membrane. The membrane can include variable concentrations of one or more pharmacotherapeutic agents for the treatment or prevention of restenosis. The membrane, in an embodiment, is made from a hydrolytically and proteolytically stable polymer, for instance, a biodurable polyurethane.

The stent of the present invention may be manufactured by initially forming a polymeric solution comprising a hydrolytically and proteolytically stable polymer. Next, at least one pharmacotherapeutic agent can be added to the polymeric solution to generate a polymer-agent mixture. Thereafter, the mixture can be applied, such as by electrostatic deposition, on to a periphery of the device in a manner which encapsulates the device. The applied mixture can then be permitted to form a porous membrane on the device. To enhance porosity, in one embodiment, the membrane can be exposed to a weak hydrochloric acid solution to allow a reaction with an alkaline metal carbonate, which can be optionally added to the polymer-agent mixture.

### BRIEF DESCIPTION OF THE DRAWINGS

FIG. 1 illustrates an encapsulated stent in accordance with an embodiment of the present invention.

FIG. 2 illustrates a side by side comparison of an encapsulated expanded stent and an unexpanded non-encapsulated stent.

FIG. 3A illustrates string-like structures within a membrane encapsulating a stent, in accordance with an embodiment of the present invention.

FIG. 3B illustrates primary micropores defined by the string-like structures in FIG. 3A within a membrane encapsulating a stent of the present invention.

FIG. 4A illustrate a secondary micropores in the membrane encapsulating a stent of the present invention.

FIG. 5 illustrates a sheet of membrane having low porosity.

FIG. 6 illustrates a graph comparing elution of a pharmacotherapeutic agent from an encapsulated stent having a high porosity membrane to a sheet of membrane having low porosity.

### DETAILED DESCRIPTION OF THE SPECIFIC EMBODIMENTS

The term “encapsulated stent”, as used hereinafter, refers to a stent in which the stent structure, both the metal mesh support and the openings defined by the struts (i.e., openings within the mesh), are completely covered with a biocompatible porous membrane. The membrane is porous and may or may not contain a pharmacotherapeutic agent.

Referring now to the drawings, FIG. I illustrates, in accordance with an embodiment of the present invention, an encapsulated stent 10 for maintaining an open lumen in a vascular structure, such as a blood vessel or an artery, and for locally delivering drug to a tissue-injured site caused by, for instance, angioplasty, where over a period of time a therapeutic dose of drug(s) may be released for the treatment of, for example, restenosis.

Previously, local drug delivery to post-angioplasty sites has been accomplished directly from an endovascular catheter. Delivery via an endovascular catheter normally involves delivering a large dose of drug in a very short time period. Because maximum benefits can be achieved by sustained drug delivery, delivery of a large dose in a short time period may not be optimal in many instances.

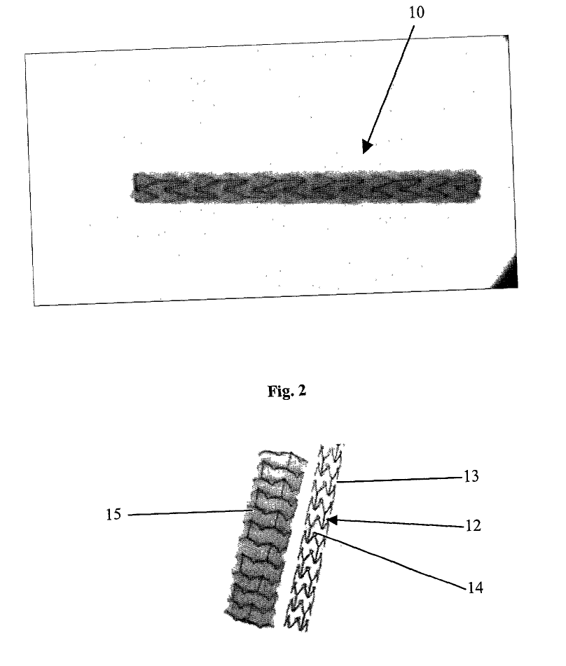

Referring now to FIG. 2, the stent 10 of the present invention, as shown on the right hand side of FIG. 2 in a relatively unexpanded state, includes a substantially cylindrical mesh support 12 having openings 13 defined by struts 14. As the stent 10 will be used to support an opening at a site which was previously closed to maintain a passage therethrough, the mesh support 12 of stent 10 needs to be made from a material that is sufficiently strong to maintain and support the opening. In addition, since the stent will be expanded when positioned at the site of interest, the material from which the stent is made also needs to be sufficiently pliable. In one embodiment of the invention, a material from which the mesh support 12 may be made includes metal.

The stent 10, as shown on the left hand side of FIG. 2 in an expanded state, further includes a coating or membrane 15 extending about a periphery of the stent 10. The extension of membrane 15 about the periphery of stent 10 also extends over the openings 13 and struts 14, so that the entire mesh structure 12 of stent 10 is substantially encapsulated by the membrane 15.

The membrane 15, in accordance with another embodiment, may also serve as a storage and direct transport vehicle for the local delivery of, for instance, restenosis-inhibiting pharmaceuticals. For use as a drug-eluting vehicle, the encapsulating membrane 15 may be made from a hydrolytically and proteolytically stable (i.e., biodurable) but porous copolymer.

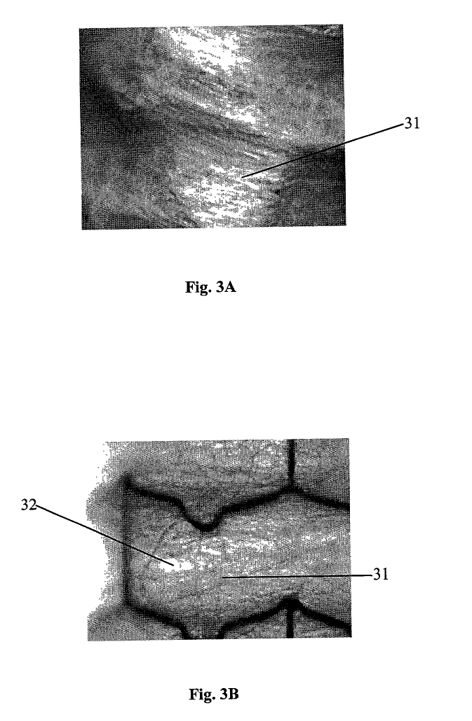

Such a copolymer, in one embodiment, may be a polycarbonate polyurethane silicon copolymer, commercially available under the trade name ChronoFlex from CardioTech International, Inc. in Woburn, Mass. The copolymer comprising the membrane 15 includes string-like structures 31, as illustrated in FIG. 3A, throughout the membrane 15, and which string-like structures 31, when overlapping one another, define micropores 32 throughout the membrane 15, as shown in FIG. 3B. The membrane 15 may also include at least one of the pharmacotherapeutic agents mentioned above incorporated or stored within the pore-defining string-like structures 31 for subsequent local delivery. An example of a pharmacotherapeutic agent which may be incorporated within the pore-defining string-like structures 31 includes Rapamycin, a phospholipid exhibiting immunosuppressive properties.

By encapsulating the stent 10 with membrane 15, and by providing porosity to membrane 15, it is believed that proper tissue (e.g., endothial cell) growth at, for example, a post-angioplasty stented site, can be enhanced.

Preparation of the membrane 15 for local delivery of a pharmacotherapeutic agent may follow the process similar to that described in U.S. Pat. No. 5,863,627 entitled, Hydrolytically-and-Proteolytically-Stable Polycarbonate Polyurethane Silicone Copolymers, and assigned to CardioTech International, Inc., Woburn, Mass. which patent is hereby incorporated herein by reference.

### EXEMPLIFICATION

The invention now being generally described, it will be more readily understood by reference to the following examples which are included merely for purposes of illustration of certain aspects and embodiments of the present invention, and are not intended to limit the invention.

Preparation of a Highly Porous Membrane with a Drug Incorporated Therein

To prepare a relatively highly porous membrane according to an embodiment of the present invention, initially, at least one pharmacotherapeutic agent, such as Rapamycin, can be dissolved at variable concentrations in one of the solvents acceptable in polymer preparation, so that the agent may be incorporated within the polymer. Examples of solvents that can be used to dissolve Rapamycin include DMSO, acetone, and chloroform. It should be appreciated that although other pharmacotherapeutic agents and more than one agent may be commercially available and suitable for treatment of restenosis, the present invention, as illustrated in the following experiments, employed the use of Rapamycin.

Subsequently, approximately seven (7) to approximately twenty (20) percent by weight of ChronoFlex, a hydrolytically and proteolytically stable porous polycarbonate polyurethane silicon copolymer, may be solubilized in di-methyl acetamide.

Thereafter, the solutions of Rapamycin and ChronoFlex may be mixed, and the resulting polymer-agent mixture is ready for application onto a stent. Application of the polymer-agent mixture may be carried out by processes known in the industry. However, in the present invention, a highly controlled process known in the industry as electrostatic deposition, and more specifically, electrostatic field assisted deposition may be employed.

To apply the polymer-agent mixture, a stent may first be placed on a rotating mandrel. The slow rotation of the mandrel, combined with a highly controlled electrostatic field assisted deposition of electrically charged droplets of the liquid polymer-agent mixture on to the stent, ensures substantially complete coverage of the stent and the openings within the mesh structure by the polymer-agent mixture. The resulting formed polymer membrane containing the pharmacotherapeutic agent is electrostatically bonded to the stent 10.

It should be noted that it is during the electrostatic field assisted deposition and the bonding process that the unique texture and primary porosity of the polymer layer/membrane is achieved. In particular, electrostatic deposition can generate a membrane having a stringlike structures 31 (See FIG. 3A), the overlapping of which generates the texture and primary porosity 32 within the membrane 15 (See FIG. 3B). As texture and porosity are deposition parameters dependent, they can therefore be varied to include a broad range of porosity. Parameters which may influence the primary porosity of the deposited polymer include the viscosity of the polymer and the deposition conditions. The deposition conditions include, the potential difference between the voltages applied to the mandrel and the spraying tip, the rotational speed of the mandrel, the distance between the mandrel and the spraying tip, and the temperature at which the deposition is taking place.

If it is desired, secondary porosity may be generated within the polymer to enhance the overall porosity of the membrane extended about the periphery of the stent. In particular, an alkali or alkali metal carbonate, such as particles of sodium carbonate porosifier, may be added to the polymer-agent mixture and stirred until uniformly dispersed before applying the mixture on to the stent. When generating secondary porosity, the micropores are generated in the body of each string-like structure themselves rather than being generated by the overlapping of the string-like structures seen with the primary porosity.

If an alkali an alkali metal porosifier has been added to the polymer-agent mixture, secondary porosity within the body of each string-like structure may be generated by soaking the polymer membrane 15 in distilled water for approximately one (1) hour or until it has absorbed water to its full capacity. Subsequently, the polymer membrane 15 may be immersed in a weak hydrochloric acid to generate a localized chemical reaction between the sodium carbonate and hydrochloric acid, which can result in the formation of water-soluble sodium chloride and carbon dioxide gas. The evolved gas escapes, while creating secondary micropores comprising a structure of interconnected tunnels and passages in the body of the string-like structure. Any entrapped sodium chloride can be washed out thereafter and the entire membrane left to dry.

Preparation of a Low Porous Membrane with a Drug Incorporated Therein

First, a pharmacotherapeutic agent, such as Rapamycin, may be dissolved at variable concentrations in one of the solvents used in polymer preparation. Next, approximately 20% by weight of ChronoFlex biostable polyurethane is solubilized in di-methyl acetamide.

Thereafter the solutions of Rapamycin and ChronoFlex may be mixed, and particles of sodium carbonate porosifier added to the polymer-agent mixture until uniformly dispersed.

The polymer-agent-porosifier mixture may subsequently be applied to a stent placed on a rotating mandrel until complete coverage of the stent and of the openings within the mesh structure is achieved. As noted above, since texture and porosity are deposition parameters dependent, deposition parameters such as rotational speed, distance along which the mixture must travel before being deposited on the stent, and voltage can be varied to generate a relatively low porosity membrane encapsulating the stent.

After the polymer membrane is deposited on to the stent, the polymer membrane may be soaked in distilled water for approximately one (1) hour or until the polymer membrane has absorbed water to its full capacity.

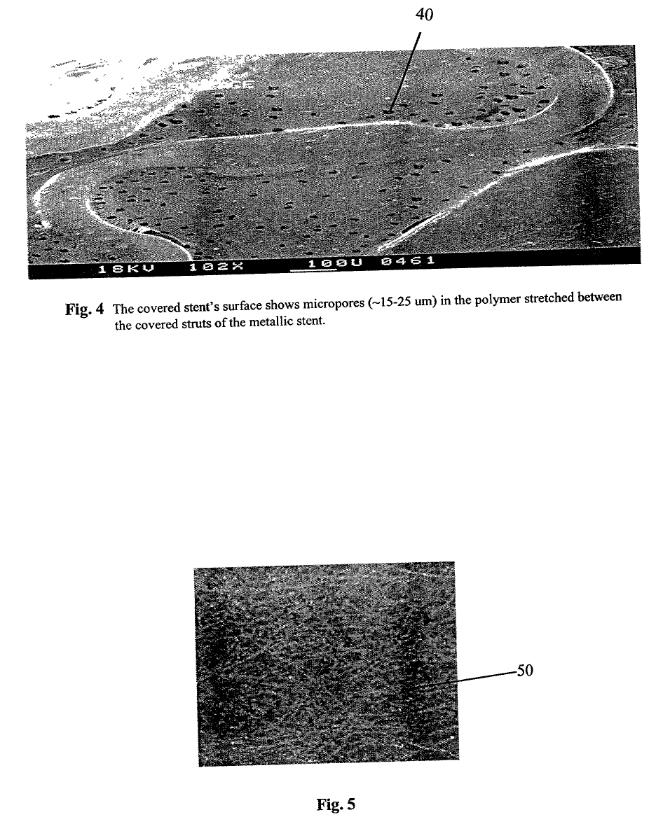

Thereafter, the waterlogged polymer membrane may be immersed in weak hydrochloric acid. A localized chemical reaction between the sodium carbonate and hydrochloric acid results in a formation of water-soluble sodium chloride and carbon dioxide gas. The evolved gas escapes, while creating a structure of interconnected tunnels and passages within the membrane. The entrapped sodium chloride is washed out and the whole structure is dried. The generated micropores 40 remain open, as shown in the scanning electron microscope photographs in FIG. 4.

Drug Delivery from a Low Porosity Polymer Membrane

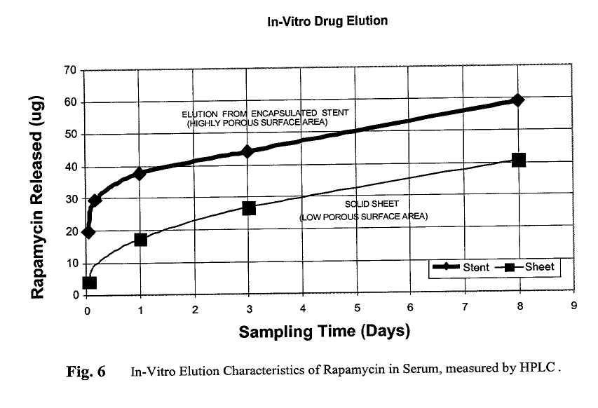

A low porosity polymer sheet 50, such as that illustrated in FIG. 5, containing approximately 14 micrograms of research grade Rapamycin per milligram of polymer was prepared according to one embodiment of the invention. Drug kinetics studies from samples containing approximately 136 micrograms of Rapamycin were conducted in calf serum and analyzed at various time intervals using HPLC. The results are shown in FIG. 6.

Drug Delivery from a High Porosity Polymer Membrane

A high porosity polymer membrane encapsulated stent containing approximately 217 micrograms of research grade Rapamycin per milligram of polymer was prepared according to an embodiment of the invention. Drug kinetics studies from unexpanded and expanded stents containing approximately 217 micrograms of Rapamycin were conducted in calf serum and analyzed at various time intervals using HPLC. The results are shown in FIG. 6.

Observations

As illustrated in FIG. 6, elution of Rapamycin over a period of several days is relatively higher in the high porosity polymer membrane. Accordingly, it can be said, by comparing the initial quantities released, that the amount of pharmacotherapeutic agent eluted can be directly proportional to the total surface from which the pharmacotherapeutic agent is eluted, and thus related the porosity and the thickness of the polymer membrane.

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

What is claimed is:

1. A device for intravascular placement, the device comprising:
a substantially cylindrical hollow body;
a membrane positioned about a periphery of the body, the membrane containing at least one pharmacotherapeutic agent for the treatment or prevention of restenosis; and
a plurality of micropores throughout the membrane.

2. A device as set forth in claim 1, wherein the body includes an expandable mesh support having openings defined by mesh support.

3. A device as set forth in claim 1, wherein the body is metallic.

4. A device as set forth in claim 1, wherein the membrane includes string-like structures defining the micropores within the membrane.

5. A device as set forth in claim 4, wherein the membrane includes additional micropores in the body of each string-like structure.

6. A device as set forth in claim 1, wherein the membrane is made from a polymer.

7. A device as set forth in claim 6, wherein the polymer is hydrolytically and proteolytically stable.

8. A device as set forth in claim 6, wherein the polymer is a biodurable polyurethane.

9. A device as set forth in claim 1, wherein the pharmacotherapeutic agent includes at least one of an immunosuppressant, an antibiotic, a cell cycle inhibitor, an anti-inflammatory, an anticoagulant, an antiallergen, and a gene therapy and a ceramide therapy compound.

10. A device as set forth in claim 1, wherein the pharmacotherapeutic agent is Rapamycin.

11. A method of manufacturing an intravascular device for local delivery of a pharmacotherapeutic agent, the method comprising:
forming a polymeric solution;
adding at least one pharmacotherapeutic agent into the polymeric solution, so as to generate a polymer-agent mixture;
applying the mixture on to a periphery of an intravascular device, so as to encapsulate the device; and
permitting a porous membrane to form from the mixture applied to the device.

12. A method as set forth in claim 11, wherein, in the step of forming, the polymeric solution comprises a hydrolytically and proteolytically stable polymer.

13. A method as set forth in claim 11, wherein the step of applying includes electrostatic field assisted depositing the mixture on to the device.

14. A method as set forth in claim 13, wherein electrostatically depositing the mixture on to the device results in the deposition of string-like structures, the overlapping of which define a primary porosity, on the resulting membrane.

15. A method as set forth in claim 11, wherein the step of adding further includes adding an alkaline metal carbonate to the polymeric solution.

16. A method as set forth in claim 15 further including exposing the membrane to a weak hydrochloric acid so as to permit a chemical reaction with the alkaline metal carbonate to generate secondary porosity in string-like structures within the membrane.

17. A method as set forth in claim 10 further including allowing the membrane to elute the pharmacotherapeutic agent in a controlled time release manner.
